(19) United States
(12) Patent Application Publication
(10) Pub. No.: US 2013/0158138 A1
(43) Pub. Date: Jun. 20, 2013

(54) INTEGRAL SYNTHESIS GAS CONVERSION CATALYST EXTRUDATES AND METHODS FOR PREPARING AND USING SAME

(71) Applicants: Kandaswamy Jothimurugesan, Hercules, CA (US); Robert J. Saxton, Pleasanton, CA (US); Howard S. Lacheen, Richmond, CA (US); Tapan Das, Albany, CA (US)

(72) Inventors: Kandaswamy Jothimurugesan, Hercules, CA (US); Robert J. Saxton, Pleasanton, CA (US); Howard S. Lacheen, Richmond, CA (US); Tapan Das, Albany, CA (US)

(73) Assignee: CHEVRON U.S.A. Inc., San Ramon, CA (US)

(21) Appl. No.: 13/327,184

(22) Filed: Dec. 15, 2011

(51) Int. Cl. C07C 27/00; B01J 37/00; B01J 29/072; B01J 29/076; B01J 29/06; B01J 29/70

(52) U.S. Cl. 518/715; 502/60; 502/74; 502/73

(57) ABSTRACT

Methods for preparing integral synthesis gas conversion catalyst extrudates including an oxide of a Fischer-Tropsch (FT) metal component and a zeolite component are disclosed. The oxide of the FT metal component is precipitated from a solution into crystallites having a particle size between about 2 nm and about 30 nm. The oxide of the FT metal component is combined with a zeolite powder and a binder material, and the combination is extruded to form integral catalyst extrudates. The oxide of the FT metal component in the resulting catalyst is in the form of reduced crystallites located outside the zeolite channels. No appreciable ion exchange of FT metal occurs within the zeolite channels. The acid site density of the integral catalyst extrudate is at least about 80% of the zeolite acid site density.

### BACKGROUND

The present disclosure relates to methods for the preparation of catalysts containing a catalytically active transition metal component and an acidic zeolite component and further relates to catalysts prepared by the methods. More particularly, the present disclosure relates to methods for the preparation of catalysts which avoid ion exchange of the transition metal component with the ions within the channels of the acidic zeolite component.

Bifunctional catalysts prepared by depositing at least one catalytically active transition metal component onto an acidic component such as a zeolite are known for use in catalytic processes such as synthesis gas conversion. Such catalysts benefit from the acid function of the zeolite, which may catalyze skeletal isomerization and cracking reactions.

Fischer-Tropsch (FT) catalysts and their preparation methods are known. FT catalysts are typically based on Group 8-10 metals such as, for example, iron, cobalt, nickel and ruthenium, also referred to herein as “FT components,” “FT active metals” or simply “FT metals,” with iron and cobalt being the most common. The product distribution over such catalysts is non-selective and is generally governed by the Anderson-Schulz-Flory (ASF) polymerization kinetics. Recent developments have led to so-called “hybrid FT” or “integral FT” catalysts having improved properties involving an FT component bound on an acidic component, typically a zeolite component. The catalytic functionality of hybrid or integral FT catalysts allows conversion of synthesis gas to desired liquid hydrocarbon products by minimizing product chain growth, thus precluding the need for further hydrocracking to obtain desired products. Thus, the combination of an FT component displaying high selectivity to short-chain α-olefins and oxygenates with zeolite(s) results in an enhanced selectivity for pourable, wax free liquid products by promoting oligomerization, cracking, isomerization, and/or aromatization reactions on the zeolite acid sites. Hybrid or integral FT catalysts for the conversion of synthesis gas to liquid hydrocarbons have been described, for example, in co-pending U.S. patent application Ser. No. 12/343,534 and U.S. Pat. No. 7,943,674 issued May, 17, 2011 (Kibby et al.), which are herein incorporated by reference.

Hybrid or integral FT catalysts are typically prepared by wet impregnation methods using aqueous or non-aqueous solutions of metal salts. During the course of this impregnation and the resultant drying and calcination, a portion of the FT metal ions (cations) migrate into the zeolite channels and essentially titrate the acid sites through ion exchange with protons in the zeolite channels. Ion exchange of the FT metal for protons within the zeolite has two disadvantages. First, zeolite acidity necessary to crack or isomerize FT olefins and to avoid making a solid wax component is neutralized. Second, ion-exchanged FT metal is non-reducible by virtue of strong metal-support interactions thus decreasing the activity of the catalyst and the overall productivity of the FT reaction. For cobalt FT metal, the ion exchange sites are quite stable positions and cobalt ions in these positions are not readily reduced during normal activation procedures. The reduction in the amount of reducible cobalt decreases the activity of the FT component in the catalyst.

A method is needed to prepare a bifunctional catalyst containing an FT metal component and an acidic component such that ion exchange of metal cations with protons within the channels of the acidic component is minimized In the resulting catalyst, both the acid capacity of the acidic component and the activity of the FT metal are maintained.

### SUMMARY

In one aspect, an integral synthesis gas conversion catalyst extrudate is provided which includes a Fischer-Tropsch component comprising an oxide of a metal selected from the group consisting of cobalt, ruthenium and mixtures thereof; a zeolite component having a zeolite acid site density; and a binder; wherein the integral synthesis gas conversion catalyst extrudate has an acid site density at least about 80% of the zeolite acid site density.

In another aspect, a method is provided for preparing the catalyst which includes the steps of forming a mixture of a Fischer-Tropsch component comprising an oxide of a metal selected from the group consisting of cobalt, ruthenium and mixtures thereof having a particle size from about 2 nm to about 30 nm, a zeolite component having a zeolite acid site density and a binder; extruding the mixture to form extrudate particles; and calcining the extrudate particles to form integral synthesis gas conversion catalyst extrudates.

In yet another aspect, a process for synthesis gas conversion is provided which includes contacting in a fixed bed reactor a synthesis gas comprising hydrogen and carbon monoxide at a ratio of hydrogen to carbon monoxide of from about 1 to about 3, at a temperature of from about 180° C. to about 280° C. and a pressure of from about 5 atmospheres to about 40 atmospheres, with the integral synthesis gas conversion catalyst extrudate, to yield a liquid hydrocarbon product containing less than about 10 weight % methane, greater than about 75 weight % C₅₊; less than about 15 weight % C₂₋₄; and less than about 5 weight % C₂₁₊ normal paraffins.

### DETAILED DESCRIPTION

In certain embodiments, the present disclosure relates to methods for the preparation of bifunctional catalysts containing at least one oxide of a Fischer-Tropsch (FT) metal and an acidic zeolite component without any appreciable ion exchange of the FT metal cations with the protons within the channels of the zeolite component. The catalyst is formed in such a way that the FT metal cations are substantially kept out of the channels of the zeolite component, thus minimizing exchange of the FT metal cations with the protons bound to the acid sites within the zeolite component.

As used herein, the terms “bifunctional catalyst” and “integral catalyst” refer interchangeably to a catalyst containing at least a catalytically active metal component and an acidic component.

The phrases “hybrid FT catalyst,” “integral FT catalyst” and “integral synthesis gas conversion catalyst” refer interchangeably to a catalyst containing an oxide of at least one FT metal component selected from the group consisting of cobalt, ruthenium and mixtures thereof, as well as an acidic component containing the appropriate functionality to convert the heavy primary C₂₁₊ products Fischer-Tropsch products into lighter, more desired products. The primary FT component is preferably cobalt.

The oxide of the at least one FT metal component to be included in the integral catalyst extrudate is formed by precipitating the metal oxide from a solution including a salt of the at least one FT metal and a precipitation agent. Preparation of the precipitation solution preferably includes mixing a compound of the FT active metal, e.g., a cobalt salt, with a solvent. The preferred solvent is water. Examples of suitable cobalt salts include, but are not limited to, cobalt nitrate, cobalt acetate, cobalt carbonyl, cobalt acetylacetonate, or the like. The FT metal component can include an optional promoter. Preparation of the precipitation solution may include mixing a compound of promoter with the solvent. Suitable promoters include platinum, palladium, rhenium, iridium, silver, copper, gold, manganese, magnesium, ruthenium, rhodium, zinc, cadmium, nickel, chromium, zirconium, cesium, lanthanum and combinations thereof.

Precipitation is preferably initiated by adding a precipitating agent to the metal salt solution prepared above. The precipitating agent can be selected from the group consisting of ammonium hydroxide, ammonium carbonate, ammonium bicarbonate, sodium hydroxide, sodium carbonate, sodium bicarbonate, potassium hydroxide, potassium carbonate and potassium bicarbonate. The pH of the solution is preferably maintained at a constant value, preferably between about 7.0 and about 10.0, while precipitation proceeds. The precipitate formed can be washed with deionized water, dried and calcined.

In one embodiment, a ruthenium promoter is included with a primary cobalt FT component in the preparation of a hybrid FT catalyst. These catalysts have very high activities due to easy activation at low temperatures. In the preparation of ruthenium promoted catalysts, any suitable ruthenium salt, such as ruthenium nitrate, chloride, acetate or the like can be used. For a catalyst containing about 10 weight % cobalt, the amount of ruthenium can be from about 0.01 to about 0.50 weight %, for example, from about 0.05 to about 0.25 weight % based upon total catalyst weight. The amount of ruthenium would accordingly be proportionately higher or lower for higher or lower cobalt levels, respectively. A catalyst level of about 10 weight % is suitable for 80 weight % ZSM-12 zeolite and 20 weight % alumina binder. The amount of cobalt can be increased as amount of alumina increases, up to about 20 weight % cobalt.

In certain embodiments, the integral FT catalyst according to the present disclosure is in the form of an extrudate containing small crystallites or particles of FT metal oxide and zeolite particles distributed in a matrix of a binder material. The combination of the zeolite powder, the FT metal oxide precipitate and the binder are formed into an integral or bifunctional catalyst extrudate by extrusion and subsequent calcination according to techniques known to those skilled in the art. The precipitated FT metal oxide as prepared above, zeolite powder and binder are mixed together with sufficient water to form a paste. The paste can then be extruded through holes in a dieplate. The integral catalyst extrudate thus formed can then be dried. The dried extrudate is then calcined by heating slowly in flowing air, for example at 10 cc/gram/minute, to a temperature in the range of from about 200° to about 800° C., even from about 300° C. to about 700° C., and even from about 400° C. to about 600° C. Calcination can be conducted by using a slow heating rate of, for example, 0.5° to about 3° C. per minute or from about 0.5° to about 1° C. per minute. The catalyst can be held at the maximum temperature for a period of about 1 to about 20 hours.

The extrudate formed can have a particle size of from about 1 mm to about 5 mm. The FT component can have a particle size from about 2 nm to about 30 nm, even from about 5 nm to about 10 nm. The zeolite component can have a particle size from about 10 nm to 10,000 nm, even from about 10 nm to about 2000 nm, and even from about 50 nm to about 500 nm. The FT metal content of the integral FT catalyst can depend on the alumina content of the zeolite. For example, for a binder content of about 20 weight % to about 99 weight % based upon the weight of the binder and zeolite, the catalyst can contain, for example, from about 1 to about 20 weight % FT metal, even 5 to about 15 weight % FT metal, based on total catalyst weight, at the lowest binder content. At the highest binder content, the catalyst can contain, for example, from about 5 to about 30 weight % FT metal, even from about 10 to about 25 weight % FT metal, based on total catalyst weight. By way of example and not limitation, suitable binder materials include alumina, silica, titania, magnesia, zirconia, chromia, thoria, boria, beryllia and mixtures thereof. The integral FT catalyst extrudate can have an external surface area of between about 10 m²/g and about 300 m²/g, a porosity of between about 30 and 80%, and a crush strength of between about 1.25 and 5 lb/mm.

Integral or bifunctional catalysts prepared according to any of the methods disclosed herein maintain full zeolite acidity after formation with the metal highly dispersed and of optimum particle size for good catalytic activity. Substantially all of the metal is in the form of reduced crystallites of metal located outside the zeolite channels with little or none of the metal located within the zeolite channels. No appreciable ion exchange of the metal therefore occurs within the zeolite channels. As a result, the percentage of residual acid sites is at least about 50%, even at least about 80%, even at least about 90%, even at least about 95% and even about 100%. As defined herein, “percentage of residual acid sites” refers to the percentage of acidity of the integral catalyst as measured by FTIR spectrometer μmol Bronsted acid sites per gram zeolite relative to the acidity of the zeolite component alone, without any additional components. In other words, the acid site density of the integral catalyst as measured by FTIR spectrometer in μmol Bronsted acid sites per gram is at least about 50%, even at least about 80%, even at least about 90%, even at least about 95% and even about 100% of the zeolite acid site density. The high percentage of residual acid sites allows for maximum utilization of metal for catalytic activity, since any metal that exchanges will not be available for catalysis.

Suitable zeolites for use in the integral catalyst include small pore molecular sieves, medium pore molecular sieves, large pore molecular sieves and extra large pore molecular sieves.

A zeolite is a molecular sieve or crystalline material having regular channels (pores) that contains silica in the tetrahedral framework positions. Examples include, but are not limited to, silica-only (silicates), silica-alumina (aluminosilicates), silica-boron (borosilicates), silica-germanium (germanosilicates), alumina-germanium, silica-gallium (gallosilicates) and silica-titania (titanosilicates), and mixtures thereof. If examined over several unit cells of the structure, the pores will form an axis based on the same units in the repeating crystalline structure. While the overall path of the pore will be aligned with the pore axis, within a unit cell, the pore may diverge from the axis, and it may expand in size (to form cages) or narrow. The axis of the pore is frequently parallel with one of the axes of the crystal. The narrowest position along a pore is the pore mouth. The pore size refers to the size of the pore mouth. The pore size is calculated by counting the number of tetrahedral positions that form the perimeter of the pore mouth. A pore that has 10 tetrahedral positions in its pore mouth is commonly called a 10 membered ring pore. Pores of relevance to catalysis in this application have pore sizes of 8 tetrahedral positions (members) or greater. If a molecular sieve has only one type of relevant pore with an axis in the same orientation to the crystal structure, it is called 1-dimensional. Molecular sieves may have pores of different structures or may have pores with the same structure but oriented in more than one axis related to the crystal.

In the acid form of a zeolite, also referred to as the H⁺ form, the acid sites are formed since a charge balancing cation is needed due the presence of aluminum in the SiO₂ framework. If the cation is a proton, as is the case for suitable zeolites for use in the present method and catalyst, the zeolite will have Bronsted acidity. The zeolite can be characterized by the density of the acid sites present in the zeolite, herein referred to as the “zeolite acid site density.”

Small pore molecular sieves are defined herein as those having 6 or 8 membered rings; medium pore molecular sieves are defined as those having 10 membered rings; large pore molecular sieves are defined as those having 12 membered rings; extra-large molecular sieves are defined as those having 14+ membered rings.

Mesoporous molecular sieves are defined herein as those having average pore diameters between 2 and 50 nm. Representative examples include the M41 class of materials, e.g. MCM-41, in addition to materials known as SBA-15, TUD-1, HMM-33, and FSM-16.

Exemplary medium pore molecular sieves include, but are not limited to, designated EU-1, ferrierite, heulandite, clinoptilolite, ZSM-11, ZSM-5, ZSM-57, ZSM-23, ZSM-48, MCM-22, NU-87, SSZ-44, SSZ-58, SSZ-35, SSZ-46 (MEL), SSZ-57, SSZ-70, SSZ-74, SUZ-4, Theta-1, TNU-9, IM-5 (IMF), ITQ-13 (ITH), ITQ-34 (ITR), and silicoaluminophosphates designated SAPO-11 (AEL) and SAPO-41 (AFO). The three letter designation is the name assigned by the IUPAC Commission on Zeolite Nomenclature.

Exemplary large pore molecular sieves include, but are not limited to, designated Beta (BEA), CIT-1, Faujasite, H—Y, Linde Type L, Mordenite, ZSM-10 (MOZ), ZSM-12, ZSM-18 (MEI), MCM-68, gmelinite (GME), cancrinite (CAN), mazzite/omega (MAZ), SSZ-26 (CON), MTT (e.g., SSZ-32, ZSM-23 and the like), SSZ-33 (CON), SSZ-37 (NES), SSZ-41 (VET), SSZ-42 (IFR), SSZ-48, SSZ-55 (ATS), SSZ-60, SSZ-64, SSZ-65 (SSF), ITQ-22 (IWW), ITQ-24 (IWR), ITQ-26 (IWS), ITQ-27 (IWV), and silicoaluminophosphates designated SAPO-5 (AFI), SAPO-40 (AFR), SAPO-31 (ATO), SAPO-36 (ATS) and SSZ-51 (SFO).

Exemplary extra large pore molecular sieves include, but are not limited to, designated CIT-5, UTD-1 (DON), SSZ-53, SSZ-59, and silicoaluminophosphate VPI-5 (VFI).

The zeolite of the catalysts of the present disclosure may also be referred to as the “acidic component” which may encompass the above zeolitic materials. The Si/A1 ratio for the zeolite can be 10 or greater, for example, between about 10 and 100. The acidic component may also encompass non-zeolitic materials such as by way of example, but not limited to, amorphous silica-alumina, tungstated zirconia, non-zeolitic crystalline small pore molecular sieves, non-zeolitic crystalline medium pore molecular sieves, non-zeolitic crystalline large and extra large pore molecular sieves, mesoporous molecular sieves and non-zeolite analogs.

According to one embodiment, the zeolite is initially in the form of a powder. Such zeolite materials can be made by known synthesis means or may be purchased.

The integral catalyst can be further activated prior to use in a synthesis gas conversion process by either reduction in hydrogen or successive reduction-oxidation-reduction (ROR) treatments. The reduction or ROR activation treatment is conducted at a temperature considerably below about 500° C. in order to achieve the desired increase in activity and selectivity of the integral catalyst. Temperatures of 500° C. or above reduce activity and liquid hydrocarbon selectivity of the catalyst. Suitable reduction or ROR activation temperatures are below 500° C., even below 450° C. and even, at or below 400° C. Thus, ranges of about 100° C. or 150° C. to about 450° C., for example, about 250° C. to about 400° C. are suitable for the reduction steps. The oxidation step should be limited to about 200° C. to about 300° C. These activation steps are conducted while heating at a rate of from about 0.1° C. to about 5° C., for example, from about 0.10° C. to about 2° C.

The catalyst can be reduced slowly in the presence of hydrogen or a mixture of hydrogen and nitrogen. Thus, the reduction may involve the use of a mixture of hydrogen and nitrogen at about 100° C. for about one hour; increasing the temperature about 0.5° C. per minute until a temperature of about 200° C.; holding that temperature for approximately 30 minutes; and then increasing the temperature about 1° C. per minute until a temperature of about 350° C. is reached and then continuing the reduction for approximately 16 hours. Reduction should be conducted slowly enough and the flow of the reducing gas maintained high enough to maintain the partial pressure of water in the offgas below 1%, so as to avoid excessive steaming of the exit end of the catalyst bed. Before and after all reductions, the catalyst should be purged in an inert gas such as nitrogen, argon or helium.

The reduced catalyst can be passivated at ambient temperature (about 25° C. to about 35° C.) by flowing diluted air over the catalyst slowly enough so that a controlled exotherm of no larger than +50° C. passes through the catalyst bed. After passivation, the catalyst is heated slowly in diluted air to a temperature of from about 300° C. to about 350° C., in the same manner as previously described in connection with calcination of the catalyst.

The temperature of the exotherm during the oxidation step should be less than about 100° C., and will be about 50° C. to about 60° C. if the flow rate and/or the oxygen concentration are dilute enough.

Next, the reoxidized catalyst is then slowly reduced again in the presence of hydrogen, in the same manner as previously described in connection with the initial reduction of the catalyst.

The combination of the FT component displaying high selectivity to short-chain α-olefins and oxygenates with the zeolite component results in an enhanced C₅₊ selectivity by promoting combinations of oligomerization, cracking, isomerization, and/or aromatization reactions on the zeolite acid sites. Desired hydrocarbon mixtures, including, for example, diesel range products, can be produced in a single reactor, e.g., a fixed bed reactor using the hybrid FT catalysts disclosed herein. Primary waxy products, when formed on the FT component, are cracked/hydrocracked by the zeolite component into mainly branched hydrocarbons with limited formation of aromatics. In particular, the presently disclosed hybrid FT catalyst can be run under certain FT reaction conditions to provide liquid hydrocarbon products containing less than about 10 weight % CH₄ and less than about 5 weight % C₂₁₊. The products formed can be substantially free of solid wax, i.e., C₂₁₊ paraffins, by which is meant that there is minimal soluble solid wax phase at ambient conditions, i.e., 20° C. at 1 atmosphere. As a result, there is no need to separately treat a wax phase in hydrocarbons effluent from a reactor.

In one embodiment, the presently disclosed hybrid FT catalyst is loaded in a fixed bed reactor, and contacted with a synthesis gas having a hydrogen to carbon monoxide ratio of from about 1 to about 3, at a temperature from about 180° C. to about 280° C. and a pressure from about 5 atmospheres to about 40 atmospheres. The resulting liquid hydrocarbon product contains less than about 10 weight % methane, greater than about 75 weight % C₅₊, less than about 15 weight % C₂₋₄, and less than about 5 weight % C₂₁₊ normal paraffins. In one embodiment, the resulting liquid hydrocarbon product has a cloud point less than about 15° C. as determined by ASTM D 2500-09.

It has been found that the reaction can be run at advantageously high pressures, such as at least about 20 atmospheres, even at least about 25 atmospheres and even at least about 30 atmospheres, thus allowing high conversion rates, while still producing a clear liquid product. By running at high pressure, the conversion process can become more economical. For instance, by running at 30 atmospheres rather than 20 atmospheres, less catalyst is required. As a consequence, the process can be run in a reactor having fewer reactor tubes loaded with catalyst.

### EXAMPLES

The methods and catalysts of the present disclosure will be further illustrated by the following examples, which set forth particularly advantageous method embodiments. While the Examples are provided to illustrate the invention, they are not intended to limit it. This application is intended to cover those various changes and substitutions that may be made by those skilled in the art without departing from the spirit and scope of the present disclosure.

### Analytical Methods

Zeolite Acidity was measured using a Nicolet 6700 FTIR spectrometer with MCT detector (available from Thermo Fisher Scientific Inc.). Materials were pressed into self supporting wafers (about 5 to about 15 mg/cm²) and degassed by heating under vacuum at about 1° C./min to about 350° C. and held at that temperature for about 1 hr before measuring spectra at about 80° C. in transmission mode. Spectra were recorded with 128 scans from about 400 to about 4000 cm⁻¹ with a resolution of about 4 cm⁻¹. Total acidity was estimated using the integrated area of acidic OH resonance centered near 3610 cm⁻¹ and correcting for the pellet weight and Co concentration.

Percentage of Residual Acid Sites was calculated by dividing the acidity measurement of an integral FT catalyst sample by the acidity measurement of the zeolite component alone. In other words, percentage of residual acid sites is the percentage of retained acidity in the integral catalyst relative to the acidity of the zeolite. For example, an extrudate consisting of about 80 wt % H-ZSM-5 and about 20 wt % Al₂O₃ would have an acidity of 100%. An integral catalyst would have an acidity of 100% if it retained all of the acid sites. The error for this measurement is less than 10% absolute.

BET surface area and pore volume of catalyst samples were determined from nitrogen adsorption/desorption isotherms measured at −196° C. using a Tristar analyzer available from Micromeritics (Norcross, Ga.). Prior to gas adsorption measurements, the catalyst samples were degassed at 190° C. for 4 hours. The total pore volume was calculated at a relative pressure of approximately 0.99.

Metal dispersion and average particle diameter were measured by hydrogen chemisorption using an AutoChem 2900 analyzer available from Micromeritics (Norcross, Ga.). The fraction of surface cobalt on catalysts was measured using H₂ temperature programmed desorption (TPD). Samples (0.25 g) were heated to 350° C. in H₂ at 1° C. min⁻¹ and held for 3 hours then cooled to 30° C. Then a flow of argon was used to purge the samples before heating to 350° C. at 20° C. min⁻¹. Hydrogen desorption was monitored using a thermal conductivity detector. TPD were repeated after oxidizing samples in 10% O₂/He and a second reduction in pure hydrogen. Dispersions were calculated relative to the cobalt concentration in each sample.

Average particle diameter of cobalt was estimated by assuming a spherical geometry of reduced cobalt. The fraction of reduced cobalt was measured by dehydrating as-prepared materials, prior to reduction, at 350° C., then cooling to room temperature and reducing in 5% H₂/Ar at a heating rate of 5° C. min-1 to 350° C. Catalyst reducibility during H₂ TPR was measured using TGA, and weight losses were assumed to be from cobalt oxide reduction in order to calculate O/Co stoichiometric ratios. The fractional reducibility was calculated by assuming the complete reduction of Co₃O₄ to Co metal, calculated using the equation below.

d(nm)=96.2*(Co Fractional Reduction)/% Dispersion

### Comparative Example 1

### Preparation of 10 wt % Co-0.25 wt % Ru/ZSM-12 by Non-Aqueous Impregnation

A catalyst containing 10 wt % Co-0.25wt % Ru on 1/16 inch (0.16 cm) alumina-bound ZSM-12 extrudates was prepared in a single step using non-aqueous impregnation. Cobalt(II) nitrate hexahydrate (available from Sigma-Aldrich, St. Louis, Mo.) and ruthenium(III) acetylacetonate (available from Alfa Aesar, Ward Hill, Mass.) were dissolved in acetone. The solution was then added to dry alumina-bound ZSM-12 extrudates. The solvent was removed in a rotary evaporator under vacuum by heating slowly to 45° C. The vacuum-dried material was then further dried in air in an oven at 120° C. overnight. The dried catalyst was then calcined at 300° C. for 2 hours in a muffle furnace. The properties of the catalyst are shown in Table 1.

### Comparative Example 2

### Preparation of 10 wt % Co-0.25wt % Ru/ZSM-12 by Aqueous Impregnation

A catalyst containing 10 wt % Co-0.25wt % Ru on 1/16 inch (0.16 cm) alumina-bound ZSM-12 extrudates was prepared in a single step using aqueous impregnation. Cobalt(II) nitrate hexahydrate (available from Sigma-Aldrich) and ruthenium(III) nitrosyl nitrate (available from Alfa Aesar) were dissolved in deionized water. The solution was then added to dry alumina-bound ZSM-12 extrudates. The excess water was removed in a rotary evaporator under vacuum by heating slowly to 60° C. The vacuum-dried material was then further dried in air in an oven at 120° C. overnight. The dried catalyst was then calcined at 300° C. for 2 hours in a muffle furnace. The properties of the catalyst are shown in Table 1.

### Example 1

To maintain the acidity of cobalt integral catalysts, the catalyst was prepared using the following method. First, a cobalt/ruthenium mixed oxide catalyst was prepared by precipitation method. Desired amounts of metal nitrates, i.e., cobalt nitrate [Co(NO₃)₂·6H₂O] and ruthenium (III) nitrosylnitrate [Ru(NO)(NO₃)₃] were dissolved in distilled water to form a solution (I). Another solution (II) was obtained by dissolving desired amount of ammonium carbonate [(NH₄)₂CO₃] in distilled water. The two solutions were simultaneously added drop wise into a beaker containing distilled water under vigorous stirring. The precipitate formed was thoroughly washed with deionized water by vacuum filtration. The wet cake of cobalt/ruthenium mixed oxide catalyst was then dried in an oven at 110° C. overnight followed by calcination at 300° C. for two hours.

Precipitated cobalt/ruthenium mixed oxide catalyst as prepared above, ZSM-12 powder (available from Zeolyst International, Conshohocken, Pa., having a SiO₂/Al₂O₃ ratio of 90) and catapal B alumina binder were added to a mixer and mixed for 15 minutes. Deionized water and a small amount of nitric acid were added to the mixed powder and mixed for additional 15 minutes. The mixture was then transferred to a 1 inch (2.54 cm) Bonnot BB Gun extruder and extruded using a 1/16″ (0.16 cm) dieplate containing 30 holes. The resulting integral catalyst extrudate was dried first at 120° C. for 2 hours and then finally calcined in flowing air at 600° C. for 2 hours. The catalyst had a composition of 10.00 wt % Co, 0.25 wt % Ru, 17.95 wt % Al₂O₃ and 71.80 wt % ZSM-12.

TABLE 1

| | FT Metal | Average | BET | Pore | Acidity |
|---|---|---|---|---|---|
| | Dispersion, | Particle | Surface | Volume, | μmol/g |
| | % | Diameter, nm | Area, m²/g | cc/g | zeolite |
| ZSM-12 | — | — | 317 | 0.445 | 253 |
| Comparative | 15.1 | 6.59 | 198 | 0.309 | 126 |
| Example 1 | | | | | |
| Comparative | 11.8 | 8.46 | 216 | 0.319 | 202 |
| Example 2 | | | | | |
| Example 1 | 10.2 | 9.74 | 283 | 0.464 | 273 |

As can be seen from the results in Table 1, the zeolite ZSM-12 was found to have an acidity of 253 μmol/g. Integral catalysts prepared by nonaqueous impregnation (Comparative Example 1) and by aqueous impregnation (Comparative Example 2) were found to have significantly lower levels of acidity. By contrast, the integral catalyst of the invention (Example 1) was found to maintain substantially all of the acidity of the zeolite. It is believed that the increase in acidity can be attributed to measurement error.

### Activation of Hybrid FT Catalysts

Fifteen grams of catalyst sample as prepared above was charged to a glass tube reactor. The reactor was placed in a muffle furnace with upward gas flow. The tube was purged first with nitrogen gas at ambient temperature, after which time the gas feed was changed to pure hydrogen with a flow rate of 750 sccm. The temperature to the reactor was increased to 350° C. at a rate of 1° C./minute and then held at that temperature for six hours. After this time, the gas feed was switched to nitrogen to purge the system and the unit was then cooled to ambient temperature. Then a gas mixture of 1 volume % O₂/N₂ was passed up through the catalyst bed at 750 sccm for 10 hours to passivate the catalyst. No heating was applied, but the oxygen chemisorption and partial oxidation exotherm caused a momentary temperature rise. After 10 hours, the gas feed was changed to pure air, the flow rate was lowered to 200 sccm and the temperature was raised to 300° C. at a rate of 1° C./minute and then kept at 300° C. for two hours. At this point, the catalyst was cooled to ambient temperature and discharged from the glass tube reactor. It was transferred to a 316-SS tube reactor of 0.51″ (1.3 cm) inner diameter and placed in a clam-shell furnace. The catalyst bed was flushed with a downward flow of helium for a period of two hours, after which time the gas feed was switched to pure hydrogen at a flow rate of 500 sccm. The temperature was slowly raised to 120° C. at a temperature interval of 1° C./minute, held there for a period of one hour, then raised to 250° C. at a temperature interval of 1° C./minute and held at that temperature for 10 hours. After this time, the catalyst bed was cooled to 180° C. while remaining under a flow of pure hydrogen gas. All flows were directed downward.

### Fischer-Tropsch Activity

The catalyst sample activated as described above was subjected to a synthesis run in which the catalyst was contacted with hydrogen and carbon monoxide at a hydrogen to carbon monoxide ratio of 2.0, at a temperature of 220° C., with a total pressure of 20-30 atm and a total gas flow rate of 2100-6000 cubic centimeters of gas (0° C., 1 atm) per gram of catalyst per hour. The results are set forth in Table 2.

TABLE 2

| | Comparative | | | |
|---|---|---|---|---|
| | Example 2 | | Example 1 | |
| TOS, h | 691 | 264 | 72 | 383 |
| Temperature,° C. | 220.0 | 220.0 | 220.0 | 220.0 |
| Pressure, atm | 20 | 30 | 20 | 30 |
| SV, mL/g/h | 3200 | 3800 | 5000 | 4150 |
| H₂/CO Fresh Feed | 2.00 | 2.00 | 2.00 | 2.00 |
| H₂ Conv., mol % | 31.4% | 41.7% | 26.7% | 33.4% |
| CO Conv, mol. % | 28.40% | 34.10% | 22.40% | 30.90% |
| Rate, gCH₂/g/h | 0.19 | 0.27 | 0.23 | 0.26 |
| Rate, mLC₅₊/g/h | 0.12 | 0.23 | 0.18 | 0.23 |
| % CO₂ | 7.10% | 0.50% | 0.40% | 0.50% |
| % CH₄ | 21.5% | 22.6% | 16.4% | 16.9% |
| % C₂ | 3.9% | 2.0% | 2.5% | 2.2% |
| % C₃ | 11.0% | 6.7 | 11.5% | 8.9% |
| % C₄ | 8.0% | 4.7% | 9.3% | 7.2% |
| % C₅₊ | 48.3% | 63.4% | 59.9% | 64.2% |
| % C₂₁₊ | 2.8% | 14.0% | 1.3% | 2.1% |
| C₂⁼/total C₂ | 37.8% | 3.5% | 10.3 | 7.2 |
| C₃⁼/total C₃ | 59.4% | 28.4% | 57.5% | 48.6% |
| C₄⁼/totalC₄ | 69.8% | 29.6% | 77.0% | 59.3% |
| Degree of Branching, % | 10.3% | 6.4% | 18.3% | 12.3% |
| Cloud Point, ° C. | 3 | >30 | 1 | 0 |
| | | (Cloudy) | | |

As can be seen from the results in Table 2, the use of an integral catalyst prepared by aqueous impregnation (Comparative Example 2) resulted in a cloudy liquid containing about 14 wt % C₂₁₊ when the process was run at 30 atmospheres pressure. By contrast, the use of the integral catalyst of the invention (Example 1) at 30 atmospheres pressure resulted in a clear liquid containing only about 2.1 wt % C₂₁₊.

Where permitted, all publications, patents and patent applications cited in this application are herein incorporated by reference, to the extent such disclosure is not inconsistent with the present invention.

Unless otherwise specified, the recitation of a genus of elements, materials or other components, from which an individual component or mixture of components can be selected, is intended to include all possible sub-generic combinations of the listed components and mixtures thereof. Also, “comprise,” “include” and its variants, are intended to be non-limiting, such that recitation of items in a list is not to the exclusion of other like items that may also be useful in the materials, compositions, methods and systems of this invention.

From the above description, those skilled in the art will perceive improvements, changes and modifications, which are intended to be covered by the appended claims.

What is claimed is:

1. An integral synthesis gas conversion catalyst extrudate comprising:
a. a Fischer-Tropsch component comprising an oxide of a metal selected from the group consisting of cobalt, ruthenium and mixtures thereof;
b. a zeolite component having a zeolite acid site density; and
c. a binder;
wherein the integral synthesis gas conversion catalyst extrudate has an acid site density at least about 80% of the zeolite acid site density.

2. The integral synthesis gas conversion catalyst extrudate of claim 1, wherein the integral synthesis gas conversion catalyst extrudate has an acid site density at least about 90% of the zeolite acid site density.

3. The integral synthesis gas conversion catalyst extrudate of claim 1, wherein the integral synthesis gas conversion catalyst extrudate has an acid site density of about 100% of the zeolite acid site density.

4. The integral synthesis gas conversion catalyst extrudate of claim 1, wherein the Fischer-Tropsch component has a particle size from about 2 nm to about 30 nm.

5. The integral synthesis gas conversion catalyst extrudate of claim 1, wherein the Fischer-Tropsch component has a particle size from about 5 nm to about 10 nm.

6. The integral synthesis gas conversion catalyst extrudate of claim 1, wherein the zeolite component is selected from the group consisting of small pore molecular sieves, medium pore molecular sieves, and large pore molecular sieves and extra large pore molecular sieves.

7. The integral synthesis gas conversion catalyst extrudate of claim 1,wherein the Fischer-Tropsch component further comprises a promoter selected from the group consisting of platinum, palladium, rhenium, iridium, silver, copper, gold, manganese, magnesium, ruthenium, rhodium, zinc, cadmium, nickel, chromium, zirconium, cesium, lanthanum and combinations thereof.

8. A method for preparing a catalyst comprising:
a. forming a mixture of a Fischer-Tropsch component comprising an oxide of a metal selected from the group consisting of cobalt, ruthenium and mixtures thereof having a particle size from about 2 nm to about 30 nm, a zeolite component having a zeolite acid site density and a binder;
b. extruding the mixture to form extrudate particles; and
c. calcining the extrudate particles to form integral synthesis gas conversion catalyst extrudates;
wherein the integral synthesis gas conversion catalyst extrudates have an acid site density of at least about 80% of the zeolite acid site density.

9. The method of claim 8, wherein the Fischer-Tropsch component is formed by precipitating a metal oxide from a solution comprising a metal selected from the group consisting of cobalt, ruthenium and mixtures thereof and a precipitation agent comprising a compound selected from the group consisting of ammonium hydroxide, ammonium carbonate, ammonium bicarbonate, sodium hydroxide, sodium carbonate, sodium bicarbonate, potassium hydroxide, potassium carbonate, and potassium bicarbonate.

10. A process for synthesis gas conversion comprising contacting in a fixed bed reactor a synthesis gas comprising hydrogen and carbon monoxide at a ratio of hydrogen to carbon monoxide of from about 1 to about 3, at a temperature of from about 180° C. to about 280° C. and a pressure of from about 5 atmospheres to about 40 atmospheres, with an integral synthesis gas conversion catalyst extrudate comprising:
a. a Fischer-Tropsch component comprising an oxide of a metal selected from the group consisting of cobalt, ruthenium and mixtures thereof having a particle size from about 2 nm to about 30 nm;
b. a zeolite component having a zeolite acid site density; and
c. a binder;
wherein the integral synthesis gas conversion catalyst extrudate has an acid site density of at least about 80% of the zeolite acid site density;
to yield a liquid hydrocarbon product containing less than about 10 weight % methane, greater than about 75 weight % C₅₊; less than about 15 weight % C₂₋₄; and less than about 5 weight % C₂₁₊ normal paraffins.

11. The process of claim 10, wherein the liquid hydrocarbon product has a cloud point less than about 15° C. as determined by ASTM D 2500-09.

12. The process of claim 10, wherein the process occurs at a pressure of at least about 25 atmospheres.

13. The process of claim 10, wherein the process occurs in pressure of at least about 30 atmospheres.
